(19) United States
(12) Patent Application Publication
(10) Pub. No.: US 2022/0102015 A1
(43) Pub. Date: Mar. 31, 2022

(54) COLLABORATIVE SMART SCREEN

(71) Applicant: GoForward, Inc., San Francisco, CA (US)

(72) Inventors: Adrian Aoun, San Francisco, CA (US); Robert Kane Sebastian, San Francisco, CA (US); Casey Edgeton, San Francisco, CA (US); Chandrashekar Raghavan, Alameda, CA (US); Alastair James Warren, San Francisco, CA (US); Sean Chin, San Francisco, CA (US); Erik Lauri Frey, Palo Alto, CA (US); Alejandra Castelao Urdaneta, Emeryville, CA (US); Ryan Christopher Oman, San Francisco, CA (US); Adam Louis Suczewski, San Francisco, CA (US); Daniel Malpas, Richmond, CA (US); Earl Taylor Roan, Castro Valley, CA (US); Ambar Ambar Choudhury, San Francisco, CA (US); James Francis Hamlin, San Francisco, CA (US)

(21) Appl. No.: 17/482,014

(22) Filed: Sep. 22, 2021

Related U.S. Application Data
(60) Provisional application No. 63/083,405, filed on Sep. 25, 2020.

(51) Int. Cl. G16H 80/00; G16H 20/10; G16H 10/40; G16H 40/60; G10L 15/26

(52) U.S. Cl. CPC G16H 80/00; G16H 20/10; G10L 15/26; G16H 40/60; G16H 10/40

(57) ABSTRACT

Systems, methods, and non-transitory media are provided for a collaborative smart screen for patient consultations. An example method can include presenting, at a display device, a suggested consultation action during a patient consultation at the medical care site, the suggested consultation action being based on patient data associated with a patient; presenting, at the display device and during the patient consultation, a portion of patient data contextually relevant to the suggested consultation action, the patient consultation, and the patient; and based on one or more measurements generated from the suggested consultation action, updating the portion of patient data presented at the display device.

### CROSS-REFERENCE TO RELATED APPLICATIONS

This application claims the benefit of priority of U.S. Provisional Application No. 63/083,405, filed on Sep. 25, 2020, entitled “COLLABORATIVE SMART SCREEN”, the contents of which are incorporated by reference in their entirety and for all purposes.

### TECHNICAL FIELD

The present disclosure generally relates to collaborative smart screens for health data and medical care.

### BACKGROUND

Health care providers use a variety of tools to provide patient care and consultations, such as health records and medical systems. In general, a patient visits a provider's office to seek a medical consultation, treatment, procedure, and care. During the patient's visit, the provider can examine the patient, issue a diagnosis, provide any treatments deemed necessary, perform any procedures deemed necessary, order tests, and prescribe medications or medical devices, among other medical tasks. The provider typically relies on information about the patient maintained in a medical system that may also be used to track patient information and provide care.

However, the information available in medical systems is often incomplete, inaccurate, and/or outdated. Moreover, medical systems used to track and access information about the patient for patient care and consultations are inefficient and generally lack an ability to collect medically-relevant data about the patient from distributed sources. Typically, most or all of the information in the medical systems is manually entered into the system and thus prone to errors or subject to missing information, and as a result such information is difficult to accurately maintain and update. Consequently, medical systems are significantly limited and can become unreliable over time.

### BRIEF SUMMARY

Disclosed are systems, methods, and computer-readable media for providing collaborative smart screens for patient care. According to at least one example, a method is provided for collaborative smart screens. The method can include presenting, at a display device at a medical care site, a suggested consultation action during a patient consultation, the suggested consultation action being based on patient data associated with a patient; presenting, at the display device and during the patient consultation, a portion of patient data contextually relevant to the suggested consultation action, the patient consultation, and the patient; and based on one or more measurements generated from the suggested consultation action, updating the portion of patient data presented at the display device.

According to at least one example, an apparatus is provided for collaborative smart screens. In some examples, the apparatus can include memory and one or more processors coupled to the memory, the one or more processors being configured to present, at a display device at a medical care site, a suggested consultation action during a patient consultation, the suggested consultation action being based on patient data associated with a patient; present, at the display device and during the patient consultation, a portion of patient data contextually relevant to the suggested consultation action, the patient consultation, and the patient; and based on one or more measurements generated from the suggested consultation action, update the portion of patient data presented at the display device.

According to at least one example, another apparatus is provided for collaborative smart screens. In some examples, the apparatus can include means for presenting, at a display device at a medical care site, a suggested consultation action during a patient consultation, the suggested consultation action being based on patient data associated with a patient; presenting, at the display device and during the patient consultation, a portion of patient data contextually relevant to the suggested consultation action, the patient consultation, and the patient; and based on one or more measurements generated from the suggested consultation action, updating the portion of patient data presented at the display device.

According to at least one example, a non-transitory computer-readable medium is provided for collaborative smart screens. The non-transitory computer-readable medium can include present, at a display device at a medical care site, a suggested consultation action during a patient consultation, the suggested consultation action being based on patient data associated with a patient; present, at the display device and during the patient consultation, a portion of patient data contextually relevant to the suggested consultation action, the patient consultation, and the patient; and based on one or more measurements generated from the suggested consultation action, update the portion of patient data presented at the display device.

In some aspects, the method, apparatuses, and non-transitory computer-readable storage medium described above can include determining an additional portion of patient data, the additional portion of patient data being based on a current context of the patient consultation; and presenting the additional portion of patient data at the display device.

In some examples, the suggested consultation action can include performing a medical test, performing a medical examination, and/or measuring a health metric via one or more medical devices. In some cases, the medical test can include a blood test, a scan, collecting and analyzing a specimen from the patient, a medical assessment, a genetic test, and/or a breathing test. In some cases, the health metric can include a blood pressure, blood glucose levels, a pulse, a body temperature, and/or a body weight.

In some aspects, the method, apparatuses, and non-transitory computer-readable storage medium described above can include receiving, from the one or more medical devices, a medical test result, a medical examination result and/or the health metric; and presenting the portion of patient data in response to receiving the medical test result, the medical examination result, and/or the health metric. In some examples, the portion of patient data can include additional patient data relevant to the suggested consultation action and the medical test result, the medical examination result, and/or the health metric. In some cases, at least part of the patient data can be received from a client device associated with the patient and/or one or more sensors at the medical care site. In some examples, the client device can include a smart phone, a sensor, a personal computer, and/or a smart wearable device, and the one or more sensors can include a wireless blood pressure sensor, a wireless heart rate sensor, a wireless body temperature sensor, a wireless pulse oximeter, a stethoscope, and/or an imaging sensor.

In some aspects, the method, apparatuses, and non-transitory computer-readable storage medium described above can include identifying an agenda for the patient consultation, the agenda being based on the patient data; and presenting, at the display device and during the patient consultation, one or more agenda items from the agenda, the one or more agenda items being associated with a context of the patient consultation. In some examples, the context can include a consultation topic, a consultation activity, and/or a health status of the patient. In some cases, the suggested consultation action can also be based on the one or more agenda items

In some aspects, the method, apparatuses, and non-transitory computer-readable storage medium described above can include determining a completion of one or more activities associated with the one or more agenda items and presenting, at the display device, one or more different agenda items. In some examples, the one or more different agenda items can be based on the patient data, additional patient data collected during the patient consultation, and/or a result of the one or more activities associated with the one or more agenda items.

In some aspects, the method, apparatuses, and non-transitory computer-readable storage medium described above can include presenting, at the display device, a transcription of speech recognized during the patient consultation; and presenting, at the display device, one or more workflow items determined for the patient consultation. In some cases, the one or more workflow items can be based on the patient data, additional patient data collected during the patient consultation, and/or the speech recognized during the patient consultation.

This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

The foregoing, together with other features and embodiments, will become more apparent upon referring to the following specification, claims, and accompanying drawings.

### BRIEF DESCRIPTION OF THE DRAWINGS

In order to describe the manner in which the various advantages and features of the disclosure can be obtained, a more particular description of the principles described above will be rendered by reference to specific embodiments thereof, which are illustrated in the appended drawings. Understanding that these drawings depict only example embodiments of the disclosure and are not to be considered to limit its scope, the principles herein are described and explained with additional specificity and detail through the use of the drawings in which:

FIG. 1 is a diagram illustrating an example system environment for patient care, in accordance with some examples of the present disclosure;

FIG. 2 is a diagram illustrating an example configuration of a collaborative smart screen, in accordance with some examples of the present disclosure;

FIG. 3 is a diagram illustrating an example use of a collaborative smart screen in a medical care site, in accordance with some examples of the present disclosure;

FIGS. 4 and 5 are diagrams illustrating example configurations of a consultation interface displayed by a collaborative smart screen, in accordance with some examples of the present disclosure;

FIG. 6 is a flowchart illustrating an example method for using a collaborative smart screen to guide a patient consultation, in accordance with some examples of the present disclosure; and

FIG. 7 illustrates an example computing device architecture, in accordance with some examples of the present disclosure.

### DETAILED DESCRIPTION

Certain aspects and embodiments of this disclosure are provided below. Some of these aspects and embodiments may be applied independently and some of them may be applied in combination as would be apparent to those of skill in the art. In the following description, for the purposes of explanation, specific details are set forth in order to provide a thorough understanding of embodiments of the application. However, it will be apparent that various embodiments may be practiced without these specific details. The figures and description are not intended to be restrictive.

The ensuing description provides example embodiments only, and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the application as set forth in the appended claims.

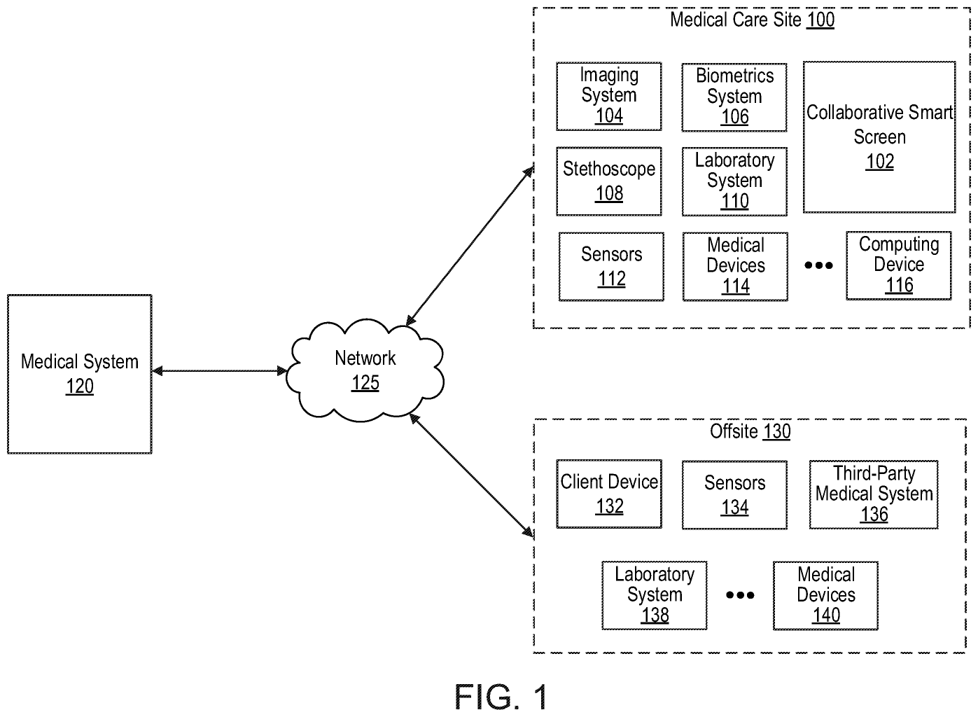

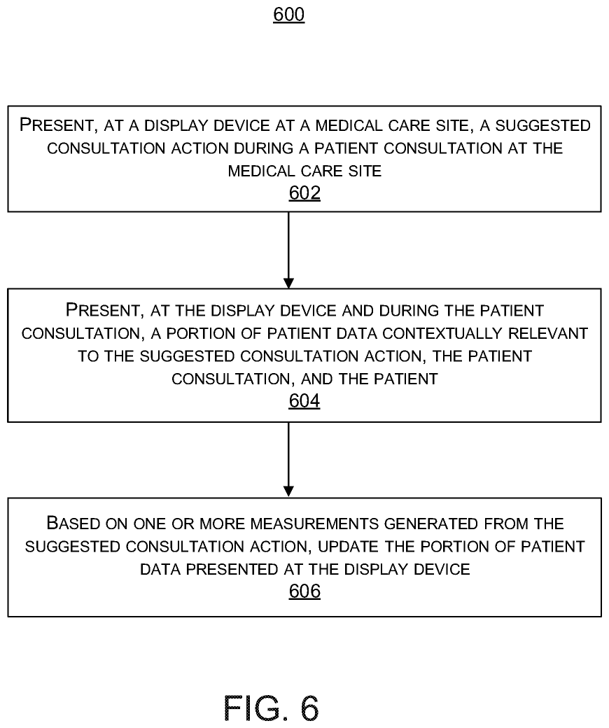

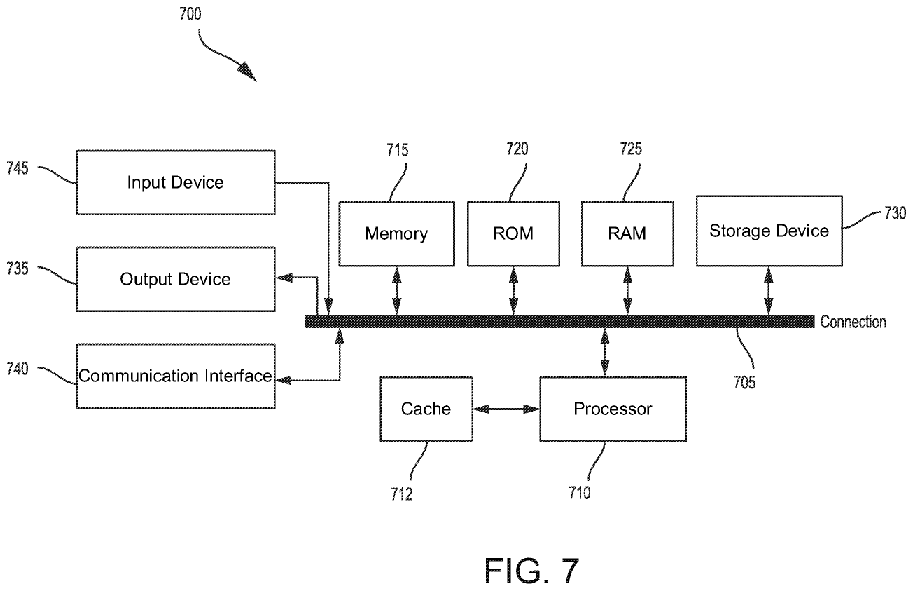

The present disclosure describes systems, methods, and computer-readable media for collaborative smart screens to guide patient consultations. The present technologies will be described in the following disclosure as follows. The discussion begins with a description of example systems, environments and technologies for providing medical care and implementing collaborative smart screens for medical consultations, as illustrated in FIG. 1 through FIG. 5. A description of an example method for implementing collaborative smart screens for patient consultations, as illustrated in FIG. 6, will then follow. The discussion concludes with a description of an example computing device architecture including example hardware components suitable for implementing medical systems, collaborative smart screens, and devices, as illustrated in FIG. 7. The disclosure now turns to FIG. 1

FIG. 1 is a diagram illustrating an example system environment for patient care. In this example, the system environment includes a medical system 120, a set of devices 102-116 in a medical care site 100, and a set of devices 132-140 at one or more offsite locations 130. However, the system environment shown in FIG. 1 is merely an illustrative example provided for explanation purposes. It should be understood that, in other examples, the system environment can include more, less, and/or different systems, devices, entities, and/or sites than those shown in FIG. 1. The medical system 120 can include one or more computing components for storing, collecting, tracking, and/or monitoring health information associated with patients. For example, the medical system 120 can include one or more computing components for storing health records, collecting health records and/or associated data and updates, providing and/or displaying health records and/or associated data, managing/maintaining scheduling information, providing notifications, providing medical requests and/or orders/prescriptions, managing health plans, etc.

In some cases, the medical system 120 can collect, store, track and monitor patient health data. The medical system 120 can collect and/or store the patient health data in encrypted form. In some examples, the patient health data can be keyed and/or correlated to the patient via one or more identifiers, such as a patient identifier. The one or more identifiers can map and/or connect to the medical records of the patient associated with the one or more identifiers. In some examples, the medical records can also be connected and/or mapped to patient credentials (e.g., login credentials) for accessing the medical system 120, medical records on the medical system 120, health tools and/or apps provided by the medical system 120, a portal hosted by the medical system 120, and/or any other features provided by the medical system 120. For example, in some cases, a patient can login to an application associated with the medical system 120 to book an appointment and/or access patient information. At the time of a booked patient visit, the application can automatically load an identifier associated with the patient, which can allow the patient's information to be automatically loaded on the medical system 120 (and/or the smart screen 102 described below). This way, the medical provider can access the patient information and does not have to manually search and retrieve the information.

The one or more computing components associated with the medical system 120 can include, for example and without limitation, one or more servers, databases, storage systems, virtual machines, software containers, datacenters, data stores, computing resources, serverless functions, cloud infrastructure, computing devices, and/or any other computing resources and/or electronic devices. [

In some cases, the medical system 120 can be located/hosted at the medical care site 100. In other cases, the medical system 120 can be located/hosted at a separate location or site. For example, the medical system 120 can be located/hosted at a separate medical care site, a location from the one or more offsite locations 130, on a cloud network, and/or on any other location.

The devices 102-116 in the medical care site 100 can include sensors and/or systems for collecting health metrics and/or performing medical tests or procedures. In FIG. 1, devices 102-116 in the medical care site 100 include a collaborative smart screen 102, one or more imaging systems 104, one or more biometric systems 106, one or more stethoscopes 108, one or more laboratory systems 110, one or more sensors 112, one or more medical devices 114, and one or more computing devices 116; and the devices 132-140 at the one or more offsite locations 130 can include one or more client devices 132, one or more sensors 134, one or more third-party medical systems 136, one or more laboratory systems 138, and one or more medical devices 140.

In some examples, the collaborative smart screen 102 in the medical care site 100 can include a smart, interactive system for dynamically displaying and providing medical information, including contextually relevant information as further described herein. The collaborative smart screen 102 can include one or more communication interfaces (e.g., wired and/or wireless) for communicating with other devices such as the medical system 120, and/or any other device

The one or more imaging systems 104 in the medical care site 100 can include one or more medical imaging and/or scanning systems such as, for example, an ultrasound system, an electrocardiogram device (ECG), a magnetic resonance imaging instrument (MRI), a computerized tomography (CT) scanner, a positron emission tomography (PET) scanner, a photoacoustic imaging device, a camera device, and/or any other imaging and/or scanning device. The one or more imaging systems 104 can include one or more communication interfaces (e.g., wired and/or wireless) for communicating test results and/or measurements to other devices such as the medical system 120, the collaborative smart screen 102, and/or any other device.

The one or more biometrics systems 106 in the medical care site 100 can include one or more biometrics sensors and/or devices such as, for example, a heart rate sensor, a blood pressure sensor, a temperature sensor, a pulse oximeter, a blood glucose sensor, a weight scale, a body composition machine/analyzer, and/or any other sensor or system for measuring biometrics. The one or more biometric systems 106 can include one or more communication interfaces (e.g., wired and/or wireless) for communicating test results and/or measurements to other devices such as the medical system 120, the collaborative smart screen 102, and/or any other device.

The one or more stethoscopes 108 in the medical care site 100 can include an electronic stethoscope. In some examples, the electronic stethoscope can include a wireless stethoscope capable of wirelessly communicating with other devices and providing measurements. The one or more stethoscopes 108 can include one or more communication interfaces (e.g., wired and/or wireless) for communicating test results and/or measurements to other devices such as the medical system 120, the collaborative smart screen 102, and/or any other device.

The one or more laboratory systems 110 and 138 can include laboratory equipment, one or more tools, and/or one or more devices for collecting, analyzing, and/or interpreting specimens such as, for example, blood samples, saliva, stool samples, urine, skin samples, etc. The one or more laboratory systems 110 and 138 can include one or more communication interfaces (e.g., wired and/or wireless) for communicating test results and/or measurements to other devices such as the medical system 120, the collaborative smart screen 102, and/or any other device.

The one or more sensors 112 and 134 can include any sensor device such as, for example, an infrared (IR) sensor, a biosensor, a tactile sensor, a pressure sensor, a respiratory sensor, a blood analyzer, a chemical sensor, an implantable sensor, a wearable sensor, a cataract sensor, a glucose meter, an activity sensor, a blood pressure sensor, a pulse oximeter, a heart rate sensor, a sleep sensor, a temperature sensor, a body composition analyzer, a stethoscope, and/or any other type of sensor. The one or more sensors 112 and 134 can include one or more communication interfaces (e.g., wired and/or wireless) for communicating test results and/or measurements to other devices such as the medical system 120, the collaborative smart screen 102, and/or any other device.

The one or more medical devices 114 and 140 can include any mechanical and/or electrical devices. For example, the one or more medical devices 114 and 140 can include a ventilator, a kidney dialysis machine, an insulin pump, a clinical bed, an anesthesia delivery machine, an oxygen concentrator, a surgical tool, a hearing test device, an ophthalmic testing device, a scope, a medicine delivery system, and/or any other medical device. The one or more medical devices 114 and 140 can include one or more communication interfaces (e.g., wired and/or wireless) for communicating test results and/or measurements to other devices such as the medical system 120, the collaborative smart screen 102, and/or any other device.

The one or more computing devices 116 and one or more client devices 132 can include a laptop computer, a desktop computer, a tablet computer, a mobile phone, an Internet-of-Things (IoT) device, a smart wearable device (e.g., a smart watch, an augmented reality device, a head-mounted display device, a smart ring, a smart meter, an activity tracker, etc.), a server, and/or any other computing device. The one or more computing devices 116 and one or more client devices 132 can include one or more communication interfaces (e.g., wired and/or wireless) for communicating test results and/or measurements to other devices such as the medical system 120, the collaborative smart screen 102, and/or any other device.

The third-party medical systems 136 can include one or more computing systems associated with one or more third parties and/or entities such as, for example, a hospital, a clinic, a doctor's office, a laboratory, a health insurance company, a health provider, etc. The third-party medical systems 136 can store, collect, track, and/or monitor health information associated with patients. For example, the third-party medical systems 136 can store and/or maintain health records, health data, medical orders, prescriptions, health metrics, medical procedure data, health statistics, health plans, patient data, etc. The third-party medical systems 136 can include one or more communication interfaces (e.g., wired and/or wireless) for communicating test results and/or measurements to other devices such as the medical system 120, the collaborative smart screen 102, and/or any other device.

As previously noted, the system environment in FIG. 1 can be used to provide medical care, consultations, and/or related services. As used herein, a “consultation” can include an onsite consultation, a remote consultation (e.g., telemedicine, etc.), or a hybrid onsite and remote consultation where one or more participants are located on site and one or more participants are located remotely. In some examples, the medical system 120, any of the set of devices 102-116 in the medical care site 100, and/or any of the set of devices 132-140 at the one or more offsite locations 130 can communicate and/or interconnect via a network 125, and can share patient and medical data. The network 125 can include one or more public and/or private networks such as, for example, one or more cloud networks, local area networks, wide area networks, virtual networks, service provider networks, core networks, datacenters, and/or the like. In some cases, the network 125 can represent the Internet.

In some examples, one or more of the devices 102-116 in the medical care site 100 can communicate and/or interconnect with one or more other devices 102-116 in the medical care site 100 directly via a peer-to-peer connection (e.g., wireless or wired) and/or via one or more networks (e.g., a wired and/or wireless local area network) on the medical care site 100. For example, in some cases, some or all of the devices 102-116 in the medical care site 100 can interconnect and/or communicate via one or more wireless connections and/or protocols (e.g., WIFI, Bluetooth, near-field communications, etc.) and/or via a local area network (LAN).

Similarly, in some examples, one or more of the devices 132-140 at the one or more offsite locations 130 can communicate and/or interconnect with one or more other devices 132-140 at the one or more offsite locations 130 directly via a peer-to-peer connection (e.g., wireless or wired) and/or via one or more networks (e.g., a wired and/or wireless local area network) at the one or more offsite locations 130. For example, in some cases, some or all of devices 132-140 that are within an offsite location can interconnect and/or communicate via one or more wireless connections and/or protocols (e.g., WIFI, Bluetooth, near-field communications, etc.) and/or via a LAN.

In some examples, the medical system 120 can collect data from one or more devices at the medical care site 100 (e.g., 102-116) and/or the one or more offsite locations 130 (e.g., 132-140). The medical system 120 can also provide data stored at the medical system 120 to one or more devices at the medical care site 100 (e.g., 102-116) and/or the one or more offsite locations 130 (e.g., 132-140).

Moreover, the collaborative smart screen 102 can send and/or receive data to/from the medical system 120 and devices 104-116 at the medical care site 100. In some cases, the collaborative smart screen 102 can also send and/or receive data to/from one or more of the devices 132-140 at the one or more offsite locations 130. For example, as further described herein, the collaborative smart screen 102 can collect data from the medical system 120 and/or any of the devices 104-116 at the medical care site 100. The collaborative smart screen 102 can use the collected data to present relevant medical and/or patient information on the collaborative smart screen 102 during a patient consultation at the medical care site 100. In some examples, the collaborative smart screen 102 can also use the collected data to prepare and/or present personalized patient health plans (e.g., treatment plans, health management plans, health monitoring plans, health goals/objectives, health programs, health schedules, actions/tasks, etc.), determine and/or present health insights, presenting and/or structuring issues, understanding a patient's body and/or physiological characteristics/conditions, etc.

In some examples, the collaborative smart screen 102 can automatically suggest personalized health plans based on inputs from the patient, health care provider, devices 104-116, devices 132-140, and/or one or more other systems, sensors, and/or users. For example, given a patient and health condition and/or goal, the collaborative smart screen 102 can suggest a set of personalized health plans based on medical best practices, choices other health care providers have made for patients with similar goals or conditions, health guidelines, and so forth. To illustrate, if a patient wants to lose weight, the collaborative smart screen 102 can suggest one or more weight loss plans that are suited to the patient's specific circumstances based on previously-prescribed plans that have shown or demonstrated efficacy for other patients. The health care provider can invoke such weight loss plan(s) and customize the weight loss plan(s) in collaboration with the patient in order to further tailor the weight loss plan(s) for the patient. For example, if the patient is vegetarian, the health care provider can customize a personalized health plan for the patient that suggests increasing protein intake by selecting one or more vegetable sources of protein.

In some cases, the collaborative smart screen 102 can learn ways to improve treatments and/or health plans for achieving certain goals for one or more patients and/or an entire patient base. For example, the collaborative smart screen 102 can learn the best and/or optimal (e.g., most effective/efficacious, top performing, etc.) treatments and/or health plans for achieving certain goals for one or more patients and/or an entire patient base. In some cases, structured issues and/or health goals can be used to represent patient health conditions and/or goals in a canonical (e.g., standard, structured, representative, normalized, unique, etc.) form to allow the collaborative smart screen 102 to learn treatments, health plans, etc., for patients. In some examples, personalized health plan suggestions can be at least partly based on structured issues and/or health goals.

In some cases, the collaborative smart screen 102 can dynamically collect, load, and/or display information during the patient consultation based on an action/task performed by the provider (e.g., a test, a measurement, an examination, a diagnosis, an input, an interaction with the patient, a question, a speech recognized by the collaborative smart screen 102, etc.), a context associated with the consultation (e.g., a reason for the consultation, a current topic of the consultation, a test and/or procedure performed and/or discussed during the consultation, biometrics associated with the patient, a condition relevant to the consultation, relevant patient information, a diagnosis associated with the consultation, a task and/or action associated with the consultation, etc.), and/or any other contextually-relevant factor.

For example, while a medical condition is addressed/discussed during the patient consultation, the collaborative smart screen can display information about the patient and relevant to the medical condition. If, while addressing the medical condition, the provider performs a test or measurement using one or more of the devices 104-116 at the medical care site 100, the collaborative smart screen 102 can dynamically collect (e.g., via push and/or pull) and display data from the test or measurement. The collaborative smart screen 102 can collect the data from the one or more of the devices 104-116 and display such data while the patient and provider address/discuss the medical condition associated with the test or measurement. If the consultation subsequently shifts to a different topic, item and/or task, the collaborative smart screen 102 similarly can dynamically collect, load, and/or display information relevant to the patient and the different topic, item, and/or task.

This way, the collaborative smart screen 102 can dynamically and intelligently gather, load, and present information relevant to a current portion of the consultation (e.g., a current topic, action, comment, etc.) and/or the consultation as a whole. As any of the devices 104-116 are used by the provider to obtain relevant patient data (e.g., test results, biometrics, measurements, etc.) during the consultation, the collaborative smart screen 102 can obtain such data and use the data to update the information presented by the collaborative smart screen 102 during the consultation. The collaborative smart screen 102 can also collect, load, and/or display relevant information obtained from other devices, such as test results, health metrics, and/or medical records from third-party systems, health metrics (e.g., measurements, statistics, test results, journal data, logged data, etc.) from one or more client devices associated with the patient (e.g., a smart watch, a heart rate sensor, a blood pressure sensor, a blood sugar sensor, a sleep sensor, an activity sensor, an image sensor, a pulse oximeter, a temperature sensor, a calorie tracker, a continuous positive airway pressure device, etc.), and/or any other device.

In some cases, the collaborative smart screen 102 can use available and/or loaded information to guide a patient consultation. For example, the collaborative smart screen 102 can dynamically display suggestions, tasks, relevant and/or contextual data, health metrics, agenda items, action items, and/or any other information tailored to allow (and/or inform) a provider to provide medical decisions and/or take actions during and/or for a patient consultation. The patient consultation can be an in person consultation or a remote consultation (e.g., telemedicine). In some examples, during a remote consultation, a patient can remotely access and/or view the content presented by the collaborative smart screen 102 (e.g., via client device 132 and network 125). In some cases, the content on the collaborative smart screen 102 can be streamed or mirrored to the patient's client device (e.g., 132) to allow the patient to view and/or interact with the content from the collaborative smart screen 102 during a remote consultation.

In some cases, the collaborative smart screen 102 can generate flags, notifications, alerts, and/or messages to identify a relevant condition, circumstance, action item, status item, attention item, and/or noteworthy item. This information can bring certain information to the attention of the health care provider and/or patient and/or trigger an action from the health care provider and/or patient. For example, if, during flu season, a patient indicates that they have not had a flu vaccine, the collaborative smart screen 102 can use such information from the patient to automatically generate a flag suggesting that a flu vaccine be administered to the patient. The health care provider can then choose to act on this suggestion by, for example, ordering the flu vaccine, scheduling the patient to receive the flu vaccine, administering the flu vaccine, etc.

As further described herein, the collaborative smart screen 102 can include artificial intelligence and/or machine learning engines for performing one or more speech, image, and/or data processing tasks. In some cases, the collaborative smart screen 102 can include a speech processing engine for analyzing and recognizing speech, and generating a transcription of recognized speech. The collaborative smart screen 102 can thus recognize, transcribe, and display speech and conversations during a patient consultation. In some cases, the collaborative smart screen 102 can also generate speech audio (e.g., via text-to-speech) to output audio instructions, suggestions, messages, notifications, and/or other utterances. By way of example, the speech processing engine may be configured to recognize speech and automatically update written patient medical records, generate patient provided health notes (e.g., to indicate patient action items or other health reminders), and/or to facilitate the generation of patient prescriptions, etc.

While the system environment in FIG. 1 is shown to include certain devices and components, one of ordinary skill will appreciate that the system environment can include more or fewer of the same and/or different devices and components than those shown in FIG. 1. For example, in some cases, the system environment can include more/less and/or different sensors, medical devices, computing devices, and/or any other systems than those shown in FIG. 1. The devices and components in FIG. 1 are merely illustrative examples provided for explanation purposes.

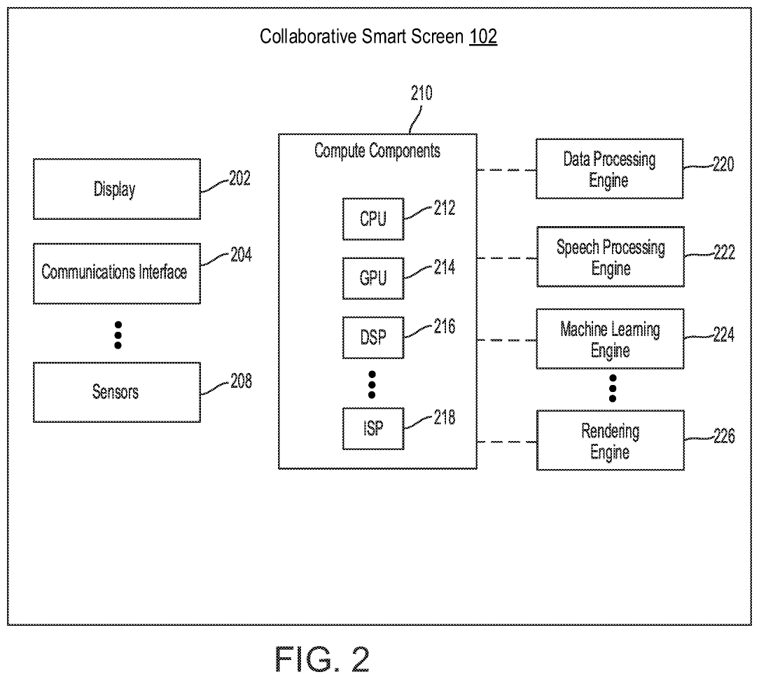

FIG. 2 is a diagram illustrating an example configuration of the collaborative smart screen 102. In this illustrative example, the collaborative smart screen 102 includes one or more displays 202, one or more communications interfaces 204 (e.g., wired and/or wireless), one or more sensors 208, compute components 210, a data processing engine 220, a speech processing engine 222, a machine learning engine 224, and a rendering engine 226. It should be noted that the components 202-226 shown in FIG. 2 are non-limiting examples provided for illustrative and explanation purposes, and other examples can include more, less, or different components than those shown in FIG. 2. For example, in some cases, the collaborative smart screen 102 can include one or more other sensors, one or more output devices, one or more input devices, one more other processing engines, one or more other hardware components, and/or one or more other software and/or hardware components that are not shown in FIG. 2. An example architecture and example hardware components that can be implemented by the collaborative smart screen 102 are further described below with respect to FIG. 7.

Moreover, references to any of the components (e.g., 202-226) of the collaborative smart screen 102 in the singular or plural form should not be interpreted as limiting the number of such components implemented by the collaborative smart screen 102 to one or more than one. For example, references to a display in the singular form should not be interpreted as limiting the number of displays implemented by the collaborative smart screen 102 to one. One of ordinary skill in the art will recognize that, for any of the components 202-226 shown in FIG. 2, the collaborative smart screen 102 can include only one of such component(s) or more than one of such component(s).

The collaborative smart screen 102 can be part of, or implemented by, a single computing device or multiple computing devices. In some examples, the collaborative smart screen 102 can be part of an electronic device (or devices) such as a display device, a computing device, etc.

In some implementations, the one or more displays 202, one or more communications interfaces 204, one or more sensors 208, compute components 210, data processing engine 220, speech processing engine 222, machine learning engine 224, and rendering engine 226 can be part of the same computing device. For example, in some cases, the one or more displays 202, one or more communications interfaces 204, one or more sensors 208, compute components 210, data processing engine 220, speech processing engine 222, machine learning engine 224, and rendering engine 226 can be integrated into a computing device. However, in some implementations, the one or more displays 202, one or more communications interfaces 204, one or more sensors 208, compute components 210, data processing engine 220, speech processing engine 222, machine learning engine 224, and rendering engine 226 can be part of two or more separate computing devices. For example, in some cases, some of the components 202-226 can be part of, or implemented by, one computing device and the remaining components can be part of, or implemented by, one or more other computing devices.

The one or more displays 202 can include any display device of any size such as, for example, a computer screen, a television display, a touch screen, and the like. For example, in some cases, the one or more displays 202 can include a large capacitive touch screen display. The large capacitive touch screen display (and the smart collaborative screen 102) can provide immersive experiences, display context information relevant to a current discussion and/or consultation, allow a health care provider to interact with the smart collaborative screen 102, and/or provide other information and/or functionalities, as further described herein.

The one or more communication interfaces 204 can include any wired and/or wireless interfaces for communicating data with other devices. In some examples, the one or more communication interfaces 204 can allow the collaborative smart screen 102 to communicate with (e.g., send and/or receive data) the medical system 120, any of the devices 104-116, any of the devices 132-140, and/or any other devices. In some cases, the one or more communication interfaces 204 can allow the smart collaboration screen 102 to connect to other devices and collect, retrieve, and/or ingest data from the other devices. The smart collaborative screen 102 can obtain such data and include the data into a patient's record and/or chart. The smart collaborative screen 102 can also process and/or visualize the data and/or a portion of the data. In some examples, the collaborative smart screen 102 can wirelessly connect (e.g., via the one or more communication interfaces 204) to one or more devices to obtain measurements, test results, and/or other data, and present such information for review by a health care provider. For example, the collaborative smart screen 102 can wirelessly connect (e.g., via the one or more communication interfaces 204) to a scanner that can image one or more parts of a patient and provide the scanned data/result to the collaborative smart screen 102. The collaborative smart screen 102 can store, process, and/or present such scanned data/results (e.g., for review by a health care provider).

The one or more sensors 208 can include any sensor device such as, for example, an image or camera sensor, an audio sensor or microphone, a tactile sensor, a pressure sensor, a light sensor, a noise sensor, a motion sensor, a proximity sensor, a gyroscope, an accelerometer, a machine vision sensor, a speech recognition sensor, a shock sensor, a position sensor, etc. In some examples, the one or more sensors 208 can include a microphone that can sense and record audio, such as voice commands. In some cases, the one or more sensors 208 can obtain voice commands, which the smart collaborative screen 102 can recognize and transcribe, as further described herein.

The one or more compute components 210 can include, for example, a central processing unit (CPU) 212, a graphics processing unit (GPU) 214, a digital signal processor (DSP) 216, and/or an image signal processor (ISP) 218. The compute components 210 can perform various operations such as graphics rendering, data processing, networking operations, image enhancement, computer vision, extended reality (e.g., tracking, localization, pose estimation, mapping, content anchoring, content rendering, etc.), image/video processing, sensor processing, recognition (e.g., text recognition, facial recognition, object recognition, feature recognition, tracking or pattern recognition, scene recognition, speech recognition, gesture recognition, etc.), machine learning, filtering, and any of the various operations described herein.

In this example, the compute components 210 implement the data processing engine 220, speech processing engine 222, machine learning engine 224, and rendering engine. In other examples, the compute components 110 can also implement one or more other processing engines. The operations for the data processing engine 220, speech processing engine 222, machine learning engine 224, and rendering engine (and any other processing engines) can be implemented by any of the compute components 210. In one illustrative example, the operations of the rendering engine 226 can be implemented by the GPU 214, and the operations of the data processing engine 220, speech processing engine 222, and/or machine learning engine 224 can be implemented by the CPU 212, the DSP 216, and/or the ISP 218. In some cases, the compute components 210 can include other electronic circuits or hardware, computer software, firmware, or any combination thereof, to perform any of the various operations described herein.

The data processing engine 220, speech processing engine 222, machine learning engine 224, and rendering engine 226 can perform respective operations based on data stored by the collaborative smart screen 102, obtained from the one or more sensors 208, and/or received from the medical system 120, one or more of the devices 104-116 at the medical care site 100, and/or one or more of the devices 132-140 at the one or more offsite locations 130. In some examples, the data processing engine 220 can process and/or analyze digital, image, and/or video data to perform calculations, generate suggestions, implement workflows, modify computer content, generate outputs, etc.

The speech processing engine 222 can process and recognize speech utterances and generate transcripts corresponding to the recognized speech. In some cases, the speech processing engine 222 can also convert text to speech to generate speech outputs based on text. In some examples, the speech processing engine 222 can include a natural language processing (NLP) system.

The rendering engine 226 can process and render data for presentation by the display 202. Moreover, the machine learning engine 224 can implement one or more neural networks and/or machine learning models to perform one or more machine learning tasks. Non-limiting examples of machine learning tasks can include computer vision, image processing, medical diagnosis, NLP, recommender systems, pattern and/or sequence analysis, health monitoring, user behavior analytics, pattern recognition, decision making, health metrics analytics, medical testing analytics, information retrieval, optimization, and the like.

In some examples, the machine learning engine 224 can be separate from the data processing engine 220, the speech processing engine 222, and/or the rendering engine 226. In other examples, the machine learning engine 224 can be part of and/or implemented by the data processing engine 220, the speech processing engine 222, and/or the rendering engine 226.

In some examples, the collaborative smart screen 102 can include one or more speakers to output sound, such as recorded sounds, speech, measured sound collected from one or more devices, etc. For example, the collaborative smart screen 102 can include one or more speakers that can play heartbeat, breathing, and/or other sounds captured by one or more sensors such as stethoscope 108.

While the collaborative smart screen 102 is shown to include certain components, one of ordinary skill will appreciate that the collaborative smart screen 102 can include more or fewer components than those shown in FIG. 2. For example, the collaborative smart screen 102 can also include, in some instances, one or more memory devices (e.g., RAM, ROM, cache, and/or the like), one or more other networking interfaces (e.g., wired and/or wireless communications interfaces and the like), one or more output and/or input devices, and/or other hardware or processing devices that are not shown in FIG. 2. An illustrative example of a computing device and hardware components that can be implemented with the collaborative smart screen 102 is described below with respect to FIG. 7.

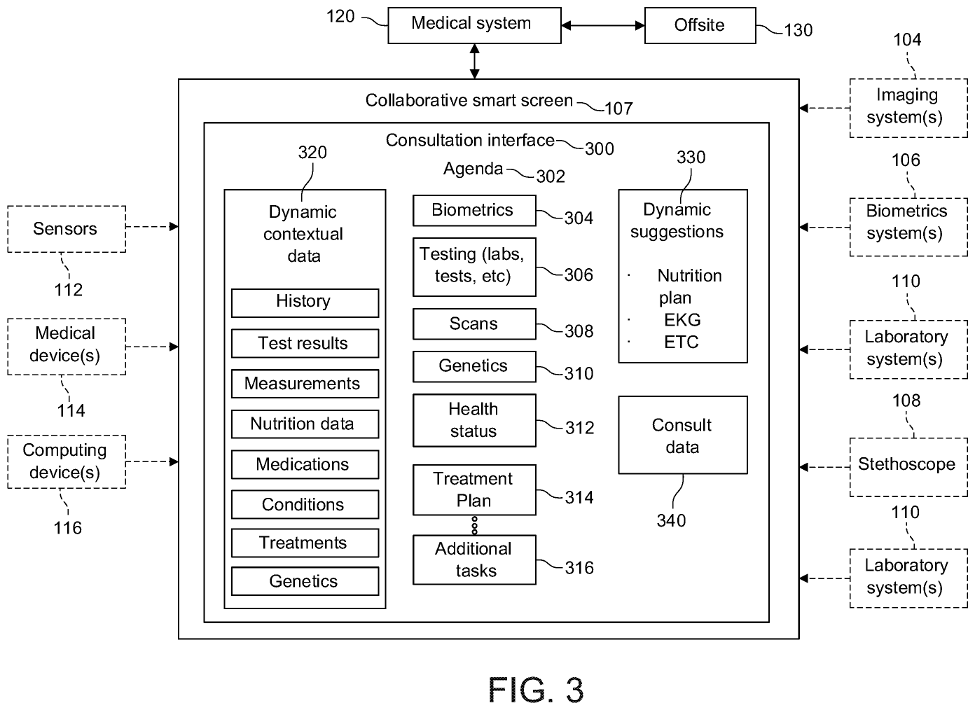

FIG. 3 is a diagram illustrating an example use of the collaborative smart screen 102 in the medical care site 100. In some examples, the collaborative smart screen 102 can be used to dynamically guide the patient consultation and provide relevant information. In some cases, during the patient consultation, the collaborative smart screen 102 can dynamically display relevant patient and/or medical data based on a current context.

For example, the collaborative smart screen 102 can dynamically load data based on an action(s) taken by the provider (e.g., an examination conducted by the provider, a test performed by the provider, a decision made by the provider, a procedure performed by the provider, a question or comment by the provider, an order/prescription issued by the provider, etc.), a topic addressed/discussed during the consultation, an issue raised during the consultation, a purpose of the consultation, information provided by the patient during the consultation, and/or any relevant event and/or circumstances. The collaborative smart screen 102 can dynamically display suggestions based on a current context and/or information associated with the patient. The suggestions can include, for example and without limitation, actions to take (e.g., orders, prescriptions, tests, procedures, examinations, referrals, treatments, questions, etc.), topics/items to address (e.g., medical issues, conditions, symptoms, diagnosis, treatment, plans, tests, etc.), issues and/or information to examine and/or verify, and/or any other activity and/or information relevant to the consultation.

In FIG. 3, the collaborative smart screen 102 is shown displaying an example consultation interface 300. The collaborative smart screen 102 can present the consultation interface 300 during a consultation (e.g., an in person or remote consultation) to help guide the consultation. In some examples, the collaborative smart screen 102 can present the consultation interface 300 to users (e.g., a patient and health care provider) during an in person consultation, allowing the users to view and/or interact with the consultation interface 300 as content is presented and/or updated. In other examples, one or more users (e.g., a patient and/or a health care provider) can access and/or interact with the consultation interface 300 remotely. For example, during a telemedicine consultation, a user can remotely connect to a consultation session hosted at the collaborative smart screen 102. The user's device can then render the consultation interface 300 for the user. The user's device can update the consultation interface rendered on the user's device as the consultation interface 300 on the collaborative smart screen 102 changes. This way, the consultation interface rendered at the user's device can remain at least partially synchronized with the consultation interface 300 presented at the collaborative smart screen 102. Other users remotely participating in the consultation can similarly access the consultation interface 300 from their device.

In some cases, the consultation interface 300 (and/or content thereof) at the collaborative smart screen 102 can be shared with, mirrored to, and/or streamed to a user's device. For example, the collaborative smart screen 102 can mirror and/or stream the consultation interface 300 (and/or content thereof) to a remote patient and/or health care provider. This way, the remote patient and/or health care provider can view and interact with the content on the collaborative smart screen 102 as it is presented and/or updated during a consultation. In some cases, the remote patient and/or health care provider can also remotely provide data to the collaborative smart screen 102, which the collaborative smart screen 102 can collect and/or use to update the content it presents/analyzes, as further described herein.

For example, if the patient remotely initiates a biometric reading (e.g., blood pressure, heart rate, temperature, etc.) at a user device, such as a smart wearable device worn by the patient, the user device can calculate the biometric information and transmit the biometric information to the collaborative smart screen 102 (e.g., via network 125). The collaborative smart screen 102 can receive the biometric information from the remote patient's device and update the consultation interface 300 based on the biometric information and/or supplement the patient's data available with the biometric information received. If the collaborative smart screen 102 updates the consultation interface 300 based on the biometric information received, the update can also be reflected on the rendered interface at the client device of any remote user participating in the consultation, such as the patient's device. Throughout the consultation, any remote users participating in the consultation can similarly access and/or interact with the consultation interface 300 (and/or content thereof) and transmit, to the collaborative smart screen 102, data remotely collected by such users (e.g., health metrics and biometric information, test data, image data, user inputs, etc.).

The consultation interface 300 in this example includes an agenda 302, dynamic contextual data 320, dynamic suggestions 330, and consultation data 340. The agenda 302 includes a biometrics interface element 304, a testing interface element 306, a scans interface element 308, a genetics interface element 310, a health status interface element 312, a treatment plan interface element 314, and an additional tasks interface element 316.

In some examples, the agenda 302 can guide the consultation by providing tasks, topics, and/or associated information to cover during the consultation. For example, the biometrics interface element 304, testing interface element 306, scans interface element 308, genetics interface element 310, health status interface element 312, treatment plan interface element 314, and additional tasks interface element 316 in the agenda 302 can indicate that the consultation should cover (e.g., review, obtain, request, and/or consider) patient biometrics, tests, scans, genetics, health status, treatment plan(s), and any additional tasks. The provider can those cover each item (e.g., 304-316) in the agenda 302 to provide a thorough, customized, and/or successful consultation. The items in the agenda 302 can be displayed in the order that they should be addressed during the consultation or any other order. In some cases, as the provider completes an item in the agenda 302, the consultation interface 300 can move and/or focus on a next item in the agenda 302. As further described below, information in the consultation interface 300 can be updated based on data and/or decisions obtained while addressing/covering that item and/or another item in the agenda 302.

In the agenda 302, the biometrics interface element 304 can represent biometrics data associated with the patient, an action item for biometrics, and/or a selectable interface object for accessing (and/or navigating to) biometrics data associated with the patient. For example, in some cases, the biometrics interface element 304 can represent biometrics information of the patient presented in the consultation interface 300. In other cases, the biometrics interface element 304 can be a label or header representing an action item for biometrics to indicate that the provider should consider, verify, measure, cover, and/or update biometrics of the patient during the consultation. In yet other cases, the biometrics interface element 304 can represent a menu for accessing biometrics of the patient. The biometrics data can include health metrics collected and/or monitored for the patient such as, for example, blood pressure, heart rate, glucose levels, body temperature, body weight, pulse oximetry, etc.

The testing interface element 306 can represent test data associated with the patient, an action item for testing, and/or a selectable interface object for accessing (and/or navigating to) test data associated with the patient. For example, in some cases, the testing interface element 306 can represent test data of the patient (e.g., previous and/or current test results) presented in the consultation interface 300. In other cases, the testing interface element 306 can be a label or header representing an action item for testing to indicate that the provider should consider, verify, cover, perform and/or update tests of the patient. In yet other cases, the testing interface element 306 can represent a menu for accessing test data of the patient. The test data can include test results collected and/or monitored for the patient such as, for example, blood tests, biopsies, saliva tests, stool tests, and/or any other medical tests.

The scans interface element 308 can represent scans associated with the patient, an action item for scans, and/or a selectable interface object for accessing (and/or navigating to) scans associated with the patient. The scans can include any scans and/or imaging results collected and/or monitored for the patient such as, for example, body scans, skin scans, CT scans, MRIs, PET scans, and/or any other medical scans.

The genetics interface element 310 can represent genetics data associated with the patient, an action item for genetics data, and/or a selectable interface object for accessing (and/or navigating to) genetics associated with the patient. The genetics data can include any genetic information, tests, and/or analysis obtained and/or monitored for the patient. Genetics information can help the patient and provider understand long-term health risks, health strategies, health insights, etc., and can be used to tailor and/or optimize health plans, treatments, and/or strategies for the patient.

The health status interface element 312 can represent health status information associated with the patient, an action item for health status, and/or a selectable interface object for accessing (and/or navigating to) health status information associated with the patient. The health status can include any information about the overall health and/or wellbeing of the patient such as, for example, health metrics, risks, conditions, normal and/or abnormal health parameters, etc.

The treatment plan interface element 314 can represent treatment plan data associated with the patient, an action item for a treatment plan, and/or a selectable interface object for accessing (and/or navigating to) treatment plan data associated with the patient. The treatment plan data can include one or more treatment plans (and associated statistics). A treatment plan can include, for example, diet, medications, procedures, lifestyle habits, care instructions, etc.

The additional tasks interface element 316 can represent data associated with additional tasks for the consultation, an action item for additional, and/or a selectable interface object for accessing (and/or navigating to) additional tasks associated with the patient. The additional tasks can include any other tasks not covered in the agenda 302 and/or resulting from other items covered in the agenda 302, such as additional tests, topics, treatments, orders, medications, examinations, protocols, instructions, procedures, checks, decisions, etc.

The dynamic contextual data 320 in the consultation interface 300 can include data dynamically loaded, displayed, and/or updated based on a current context of the consultation. For example, the dynamic contextual data 320 can include medical history information, test results, measurements, nutrition data, medications, conditions, treatments, genetics, etc., that is/are relevant to a current agenda item (e.g., 304-316) being covered, a current topic being covered, a current action being performed (e.g., a current test, examination, procedure, etc.), a current decision being made by the provider, and/or any other current circumstances.

The dynamic suggestions 330 in the consultation interface 300 can include suggestions dynamically generated, displayed, and/or updated based on a current context of the consultation. For example, the dynamic suggestions 330 can include suggested tests, measurements, diet plans, medications, treatments, procedures, examinations, orders, actions, etc. In some examples, such suggestions can be generated, displayed, and/or updated based on a current agenda item (e.g., 304-316) being covered, a current topic being covered, a current action being performed (e.g., a current test, examination, procedure, etc.), a current decision being made by the provider, patient data previously obtained and/or determined, patient data obtained and/or determined during the consultation, and/or any other relevant information.

The consultation data 340 can include data generated during the consultation. For example, the consultation data 340 can include a transcription of some or all discussions/speech during the consultation, notes generated during the consultation, orders generated during the consultation, prescriptions created during the consultation, etc.

In some examples, the agenda 302, dynamic contextual data 320, dynamic suggestions 330, and/or consultation data 340 (and/or any associated data) can be determined, loaded and displayed in the consultation interface 300 dynamically based on data associated with the patient and/or the consultation received (e.g., wirelessly and/or via a wired network connection) from the medical system 120, one or more systems (e.g., 104-116) in the medical care site 100 and/or one or more devices (e.g., 132-140) in the one or more offsite locations 130. For example, before and/or during the consultation, the collaborative smart screen 102 can receive from the medical system 120 data relevant to the patient and consultation such as a medical record of the patient. The collaborative smart screen 102 can use the data received to determine some or all of the data initially presented in the consultation interface 300.

In some cases, the agenda 302, dynamic contextual data 320, dynamic suggestions 330, consultation data 340 and/or any associated data can be updated as new data is received from the medical system 120 and/or one or more systems in the medical care site 100. For example, the provider can use the sensors 112 to measure biometrics of the patient, such as a heart rate, blood pressure, weight, blood glucose levels, oxygen levels, etc. The collaborative smart screen 102 can then receive (e.g., via a wired and/or wireless transmission) the measured biometrics from the sensors 112, and update the agenda 302, dynamic contextual data 320, dynamic suggestions 330, consultation data 340 and/or any associated data.

If the provider uses the one or more imaging systems 104 to obtain a scan (e.g., a body scan, a skin scan, a CT scan, etc.) for the patient during the consultation, the collaborative smart screen 102 can receive the scan from the one or more imaging systems 104 and similarly update the agenda 302, dynamic contextual data 320, dynamic suggestions 330, consultation data 340 and/or any associated data. In this way, any data (e.g., measurements, outputs, results, etc.) collected by the provider using any of the devices 104-116 in the medical care site 100 can be dynamically loaded and displayed on the collaborative smart screen 102 and/or used to dynamically update content presented by the collaborative smart screen 102 (e.g., the agenda 302, dynamic contextual data 320, dynamic suggestions 330, consultation data 340 and/or any associated data).

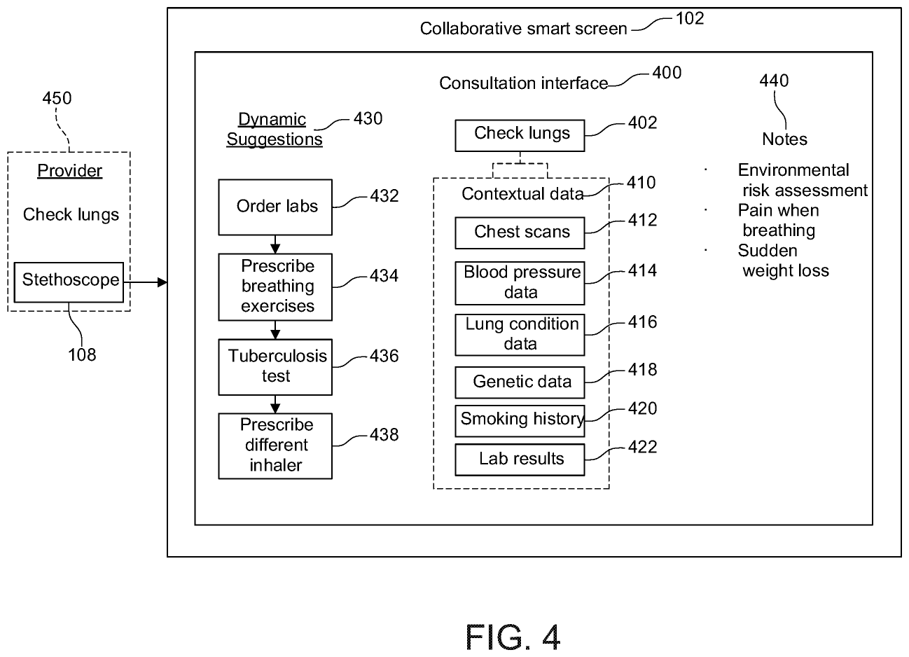

FIG. 4 is a diagram illustrating an example configuration of a consultation interface 400 displayed by the collaborative smart screen 102. In this example, the consultation interface 400 displays a check lungs action 402 for the provider to perform during the consultation. In some cases, the check lungs action 402 can be a standing and/or predetermined action included in the current and/or all consultations. In other cases, the check lungs action 402 can be an action specifically defined and/or tailored for the patient. For example, if the patient has a lung condition or is at risk for a lung condition, the consultation interface 400 can display the check lungs action 402 to indicate that the patient's lungs should be checked and/or certain actions to check the lungs should be performed. In some examples, the check lungs action 402 can indicate that a general lung check or general lung health status should be performed. In other examples, the check lungs action 402 can indicate specific lung health indicators and/or conditions to be checked, specific evaluations, specific tests, specific metrics, specific genetic factors, specific symptoms, and/or any other specific actions and/or factors to check.

The check lungs action 402 can be presented along with contextual data 410, which can include data (previously and/or currently obtained) relevant to the check lungs action 402. For example, the contextual data 410 can include patient chest scans 412, blood pressure data 414, lung condition data 416, genetic data 418, smoking history 420, and lab results 422. It should be noted that the contextual data items 412-422 in the contextual data 410 shown in FIG. 4 are non-limiting illustrative examples provided for explanation purposes. Other examples may include more/less and/or different contextual data and/or related items.

The contextual data 410 can provide the provider information relevant to the check lungs action 402. For example, the contextual data 410 can provide data that can help the provider perform the check lungs action 402, indicate (or help the provider understand) what actions to take to check for lung health, indicate (or help the provider understand) what to look for or consider when performing the check lungs action 402, indicate (or help the provider understand) options and/or instructions for checking the lungs, etc.

The contextual data 410 and/or contextual data items 412-422 can include data generated, obtained, and/or taken previous to the consultation. The contextual data 410 and/or contextual data items 412-422 can also include data generated, collected, and/or taken during the consultation. For example, based on the check lungs action 402, the provider can perform a lung examination 450 using the stethoscope 108. The stethoscope 108 can transmit measurements and/or other data produced during the lung examination 450 to the collaborative smart screen 102, which can use such data from the stethoscope 108 to dynamically update the contextual data 410. The data from the stethoscope 108 can be used to update an existing portion of the contextual data 410, such as the lung condition data 416 for example, and/or to add or create new contextual data or a new portion of contextual data, which can be included as part of the contextual data 410. In other words, the data from the stethoscope 108 can be used to update existing information in the contextual data 410 and/or expand the contextual data 410 to include new or additional information.

For example, the stethoscope 108 can send to the collaborative smart screen 102 a measured or recorded sound of the heart and lungs. The collaborative smart screen 102 can analyze (e.g., via the data processing engine 220 and/or the machine learning engine 224) the acoustic properties of the sound to determine certain characteristics and/or conditions of the lungs. To illustrate, based on the acoustic properties of the sound, the collaborative smart screen 102 can identify absent or decreased breathing sounds and/or abnormal breathing sounds. In some examples, the absent or decreased sounds can be used to infer that there is air or fluid in or around the lungs, which can indicate certain conditions such as pneumonia, heart failure, pleural effusion, etc.; increased thickness of the chest wall; reduced airflow to the lungs (or a portion of the lungs); over-inflation of a part of the lungs, which can indicate certain conditions such as emphysema; etc. Moreover, abnormal breathing sounds can be used to infer a variety of conditions such as, for example, asthma, bronchitis, chronic obstructive pulmonary disease (COPD), allergies, etc.

In this way, the collaborative smart screen 102 can determine certain characteristics and/or conditions of the lungs based on the data received from the stethoscope 108, and update the contextual data 410 to include such characteristics and/or conditions of the lungs. In the example shown in FIG. 4, the collaborative smart screen 102 can, for example, update the lung condition data 416 to include the characteristics and/or conditions of the lungs determined from the stethoscope data and/or add new lung condition data in the contextual data 410 to include the characteristics and/or conditions of the lungs determined from the stethoscope data. If additional measurements are subsequently obtained using other devices in the medical care site 100, such additional measurements (and/or determinations made based on such additional measurements) can be similarly used to update the contextual data 410. The contextual data 410 can thus dynamically evolve with the consultation and/or reflect new information and/or insights gained during the consultation.

The consultation interface 400 can also include dynamic suggestions 430 and consultation notes 440. The dynamic suggestions 430 can be contextually related to the check lungs action 402 and/or an action taken by the provider (e.g., lung examination 450). In this illustrative example, the dynamic suggestions 430 include a sequence of actions 432-438 suggested dynamically based on the check lungs action 402, the contextual data 410, the lung examination 450, and/or any other relevant data. The actions 432-438 in this example include ordering labs 432 (e.g., blood work, lung tests, etc.), prescribing breathing exercises 434, performing a tuberculosis test 436, and prescribing a different inhaler 438.

In some cases, the actions 432-438 can be dynamically determined using a machine learning algorithm (e.g., via machine learning engine 224). In some cases, the actions 432-438 can be determined based on a template list of actions selected when one or more criteria for the template list of actions are satisfied. For example, a template list of actions for checking the lungs can indicate that actions 432-438 should be performed when certain risk factors and/or lung conditions are satisfied. Thus, when the collaborative smart screen 102 determines that the risk factors and/or lung conditions associated with that template are satisfied, the collaborative smart screen 102 can determine that the actions (e.g., 432-438) in the template should be performed. The collaborative smart screen 102 can dynamically display the actions 432-438 based on the template and determination.

In some cases, as other relevant actions are performed during the consultation and/or other relevant data collected during the consultation, the collaborative smart screen 102 can use such data to dynamically update the contextual data 410 and/or the dynamic suggestions 430. When the check lungs action 402 is complete, the collaborative smart screen 102 can present a next action to be performed, which can be a predetermined or dynamically determined action, or indicate that the consultation is complete if such is the case.

In some examples, the consultation interface 400 can display notes 440 from the consultation. In some cases, the notes 440 can include a transcription of the consultation. Moreover, the notes 440 can include provider notes and/or other data collected, entered, and/or generated during the consultation. The notes 440 can be displayed on the consultation interface 400 dynamically as the associated data is generated, collected, etc. In some cases, some or all of the data in the notes 440 can be manually entered via the collaborative smart screen 102 and/or one or more separate computing devices, such as computing device 116 or medical system 120.

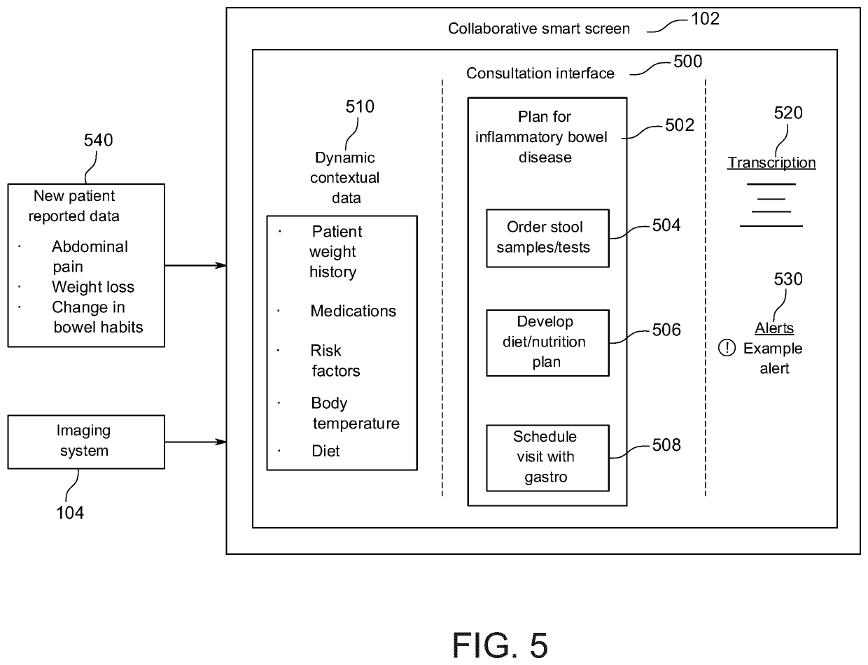

FIG. 5 is a diagram illustrating another example configuration of a consultation interface 500 displayed by the collaborative smart screen 102. In this example, the consultation interface 500 includes a plan for inflammatory bowel disease 502. The plan 502 can identify steps 504-508 to guide a consultation relating to a patient diagnosed with, at risk of, or being screened for inflammatory bowel disease. The consultation interface 500 also includes dynamic contextual data 510, a transcription 520 of the consultation, and alerts 530 (e.g., messages, notifications, etc.) generated during the consultation.

The steps 504-508 in the plan 502 include ordering a stool sample and test 504, developing a diet plan 506, and scheduling 508 a visit with a gastroenterologist. The steps 504-508 can include predetermined actions for inflammatory bowel disease consultations and/or actions tailored and/or specifically determined for the current patient and inflammatory bowel disease consultation based on patient data obtained before and/or during the consultation.

In some examples, some or all of the plan 502, dynamic contextual data 510, and alerts 530 can be dynamically generated and/or updated based on actions and data from the consultation. For example, some or all of the plan 502, dynamic contextual data 510, and alerts 530 can be dynamically generated and/or updated based on new patient data 540 reported during the consultation and scan results obtained and received from the one or more imaging systems 104 during the consultation.

Moreover, the transcription 520 of the consultation can be generated dynamically during the consultation. For example, in some cases, the speech processing engine 222 can recognize and transcribe speech during the consultation. The collaborative smart screen 102 can then present the transcribed speech as it is generated.

Having disclosed example systems, components and concepts, the disclosure now turns to the example method 600 for using a collaborative smart screen (e.g., 102) to guide a patient consultation, as shown in FIG. 6. The steps outlined herein are non-limiting examples provided for illustration purposes, and can be implemented in any combination thereof, including combinations that exclude, add, or modify certain steps.

At block 602, the method 600 can include presenting, at a display device (e.g., collaborative smart screen 102) at a medical care site (e.g., 100), a suggested consultation action (e.g., 302, 304-316, 330, 402, 432-438, 502, 504-508) during a patient consultation. Users (e.g., a patient, a health care provider, a parent, etc.) participating in the patient consultation can be located on site and/or at a remote location. Moreover, in some cases, the patient consultation can be at the medical care site. In other cases, the patient consultation can be a telemedicine consultation where all or some users are participating remotely. In some examples, the suggested consultation action presented at the display device (and/or any other content presented at the display device during the patient consultation) can also be presented (e.g., streamed, mirrored, shared, etc.) at a remote user's device.

In some examples, the suggested consultation action can be based on patient data associated with a patient. For example, the suggested consultation action can be a consultation action (e.g., a test, an examination, a health metric measurement, a scan, a procedure, a treatment, an order, a prescription, a screening, a physical, etc.) determined based on a medical record of the patient, patient information collected during the patient consultation, and/or one or more health metrics (e.g., test results, biometrics, scans, examination results, etc.) generated/obtained during the patient consultation.

In some examples, the suggested consultation action can include performing a medical test, performing a medical examination, and/or measuring a health metric via one or more medical devices (e.g., 104-116) at the medical care site. In some cases, the medical test can include a blood test, a scan, collecting and analyzing a specimen (e.g., blood, saliva, stool, a skin sample, etc.) from the patient, a medical assessment, a genetic test, and/or a breathing test. In some cases, the health metric can include a blood pressure, blood glucose levels, a pulse, a body temperature, and/or a body weight.

In some cases, at least part of the patient data is received from a client device (e.g., 132) associated with the patient and/or one or more sensors (e.g., 104, 106, 108, 112, etc.) at the medical care site. In some examples, the client device can include a smart phone and/or a smart wearable device (e.g., a smart watch, an activity tracker, a smart ring, a portable sensor, a pulse oximeter, a blood pressure monitor, a sleep monitor, etc.), and the one or more sensors can include a wireless blood pressure sensor, a wireless heart rate sensor, a wireless body temperature sensor, a wireless pulse oximeter, a stethoscope, and/or an imaging sensor (e.g., a scanner, a camera, etc.).

At block 604, the method 600 can include presenting, at the display device and during the patient consultation, a portion of patient data (e.g., 320, 410, 412-422, 510) contextually relevant to the suggested consultation action, the patient consultation, and the patient. In some examples, the portion of patient data can include a patient health status, information from a patient medical record, measurements and/or metrics collected through a previous consultation action, etc.

At block 606, the method 600 can include based on one or more measurements (e.g., health metrics, scans, test results, biometrics, etc.) generated from the suggested consultation action, updating the portion of patient data presented at the display device. In some examples, the method 600 can include receiving the one or more measurements from one or more devices at the medical care site and dynamically updating a presentation at the display device based on the one or more measurements.

In some aspects, the method 600 can include receiving, from the one or more medical devices, a medical test result, a medical examination result and/or the health metric, and presenting the portion of patient data in response to receiving the medical test result, the medical examination result, and/or the health metric. In some examples, the portion of patient data can include additional patient data relevant to the suggested consultation action and the medical test result, the medical examination result, and/or the health metric.

In some aspects, the method 600 can include determining an additional portion of patient data, and presenting the additional portion of patient data at the display device. In some examples, the additional portion of patient data can be based on a current context of the patient consultation and/or the patient data.

In some aspects, the method 600 can include identifying an agenda for the patient consultation, and presenting, at the display device and during the patient consultation, one or more agenda items from the agenda. In some cases, the agenda can be based on the patient data, and the one or more agenda items can be associated with (e.g., relevant to, based on, etc.) a context of the patient consultation. In some cases, the context can include a consultation topic, a consultation activity, and/or a health status of the patient. In some cases, the suggested consultation action can be further based on the one or more agenda items.

In some aspects, the method 600 can include determining a completion of one or more activities associated with the one or more agenda items; and presenting, at the display device, one or more different agenda items. In some examples, the one or more different agenda items can be based on the patient data, additional patient data collected during the patient consultation, and/or a result of the one or more activities associated with the one or more agenda items.

In some aspects, the method 600 can include presenting, at the display device, a transcription (e.g., 520) of speech recognized during the patient consultation; and presenting, at the display device, one or more workflow items (e.g., agenda items, actions, etc.) determined for the patient consultation. In some examples, the one or more workflow items can be based on the patient data, additional patient data collected during the patient consultation, and/or the speech recognized during the patient consultation.

In some examples, the method 600 may be performed by one or more computing devices or apparatuses. In one illustrative example, the method 600 can be performed by the collaborative smart screen 102 shown in FIGS. 1 and 2 and/or one or more computing devices with the computing device architecture 700 shown in FIG. 7. In some cases, such a computing device or apparatus may include a processor, microprocessor, microcomputer, or other component of a device that is configured to carry out the steps of the method 600. In some examples, such computing device or apparatus may include one or more sensors configured to capture image data. For example, the computing device can include a smartphone, a head-mounted display, a mobile device, a display screen, or other suitable device. In some examples, such computing device or apparatus may include a display configured to display computer data and/or graphics. In some cases, such computing device may include a display for displaying digital data.

The components of the computing device can be implemented in circuitry. For example, the components can include and/or can be implemented using electronic circuits or other electronic hardware, which can include one or more programmable electronic circuits (e.g., microprocessors, graphics processing units (GPUs), digital signal processors (DSPs), central processing units (CPUs), and/or other suitable electronic circuits), and/or can include and/or be implemented using computer software, firmware, or any combination thereof, to perform the various operations described herein. The computing device may include a display (as an example of the output device or in addition to the output device), a network interface configured to communicate and/or receive the data, any combination thereof, and/or other component(s). The network interface may be configured to communicate and/or receive Internet Protocol (IP) based data or other type of data.

The method 600 is illustrated as a logical flow diagram, the operations of which represent a sequence of operations that can be implemented in hardware, computer instructions, or a combination thereof. In the context of computer instructions, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Additionally, the method 600 may be performed under the control of one or more computer systems configured with executable instructions and may be implemented as code (e.g., executable instructions, one or more computer programs, or one or more applications) executing collectively on one or more processors, by hardware, or combinations thereof. As noted above, the code may be stored on a computer-readable or machine-readable storage medium, for example, in the form of a computer program comprising a plurality of instructions executable by one or more processors. The computer-readable or machine-readable storage medium may be non-transitory.

FIG. 7 illustrates an example computing device architecture 700 of an example computing device which can implement various techniques described herein. For example, the computing device architecture 700 can implement at least some portions of the medical system 120 shown in FIG. 1 and/or the collaborative smart screen 102 220 shown in FIGS. 1 and 2. The components of the computing device architecture 700 are shown in electrical communication with each other using a connection 705, such as a bus. The example computing device architecture 700 includes a processing unit (CPU or processor) 710 and a computing device connection 705 that couples various computing device components including the computing device memory 715, such as read only memory (ROM) 720 and random access memory (RAM) 725, to the processor 710.

The computing device architecture 700 can include a cache of high-speed memory connected directly with, in close proximity to, or integrated as part of the processor 710. The computing device architecture 700 can copy data from the memory 715 and/or the storage device 730 to the cache 712 for quick access by the processor 710. In this way, the cache can provide a performance boost that avoids processor 710 delays while waiting for data. These and other modules can control or be configured to control the processor 710 to perform various actions. Other computing device memory 715 may be available for use as well. The memory 715 can include multiple different types of memory with different performance characteristics. The processor 710 can include any general purpose processor and a hardware or software service stored in storage device 730 and configured to control the processor 710 as well as a special-purpose processor where software instructions are incorporated into the processor design. The processor 710 may be a self-contained system, containing multiple cores or processors, a bus, memory controller, cache, etc. A multi-core processor may be symmetric or asymmetric.

To enable user interaction with the computing device architecture 700, an input device 745 can represent any number of input mechanisms, such as a microphone for speech, a touch-sensitive screen for gesture or graphical input, keyboard, mouse, motion input, speech and so forth. An output device 735 can also be one or more of a number of output mechanisms known to those of skill in the art, such as a display, projector, television, speaker device. In some instances, multimodal computing devices can enable a user to provide multiple types of input to communicate with the computing device architecture 700. The communication interface 740 can generally govern and manage the user input and computing device output. There is no restriction on operating on any particular hardware arrangement and therefore the basic features here may easily be substituted for improved hardware or firmware arrangements as they are developed.

Storage device 730 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 725, read only memory (ROM) 720, and hybrids thereof. The storage device 730 can include software, code, firmware, etc., for controlling the processor 710. Other hardware or software modules are contemplated. The storage device 730 can be connected to the computing device connection 705. In one aspect, a hardware module that performs a particular function can include the software component stored in a computer-readable medium in connection with the necessary hardware components, such as the processor 710, connection 705, output device 735, and so forth, to carry out the function.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Specific details are provided in the description above to provide a thorough understanding of the embodiments and examples provided herein. However, it will be understood by one of ordinary skill in the art that the embodiments may be practiced without these specific details. For clarity of explanation, in some instances the present technology may be presented as including individual functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual embodiments may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code. Examples of computer-readable media that may be used to store instructions, information used, and/or information created during methods according to described examples include magnetic or optical disks, flash memory, USB devices provided with non-volatile memory, networked storage devices, and so on.

Devices implementing processes and methods according to these disclosures can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Typical examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

In the foregoing description, aspects of the application are described with reference to specific embodiments thereof, but those skilled in the art will recognize that the application is not limited thereto. Thus, while illustrative embodiments of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

One of ordinary skill will appreciate that the less than (“<”) and greater than (“>”) symbols or terminology used herein can be replaced with less than or equal to (“≤”) and greater than or equal to (“≥”) symbols, respectively, without departing from the scope of this description.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” refers to any component that is physically connected to another component either directly or indirectly, and/or any component that is in communication with another component (e.g., connected to the other component over a wired or wireless connection, and/or other suitable communication interface) either directly or indirectly.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

The various illustrative logical blocks, modules, circuits, and algorithm steps described in connection with the examples disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods, algorithms, and/or operations described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein.

What is claimed is:

1. A method comprising:
presenting, at a display device at a medical care site, a suggested consultation action during a patient consultation, the suggested consultation action being based on patient data associated with a patient;
presenting, at the display device and during the patient consultation, a portion of patient data contextually relevant to the suggested consultation action, the patient consultation, and the patient; and
based on one or more measurements generated from the suggested consultation action, updating the portion of patient data presented at the display device.

2. The method of claim 1, wherein the suggested consultation action comprises at least one of performing a medical test, performing a medical examination, and measuring a health metric via one or more medical devices.

3. The method of claim 2, wherein the medical test comprises at least one of a blood test, a scan, collecting and analyzing a specimen from the patient, a medical assessment, a genetic test, and a breathing test.

4. The method of claim 2, wherein the health metric comprises at least one of blood pressure, blood glucose levels, a pulse, a body temperature, and a body weight.

5. The method of claim 2, further comprising:
receiving, from the one or more medical devices, at least one of a medical test result, a medical examination result and the health metric; and
presenting the portion of patient data in response to receiving the at least one of the medical test result, the medical examination result, and the health metric.

6. The method of claim 5, wherein the portion of patient data comprises additional patient data relevant to the suggested consultation action and the at least one of the medical test result, the medical examination result, and the health metric.

7. The method of claim 1, further comprising:
determining an additional portion of patient data, the additional portion of patient data being based on a current context of the patient consultation; and
presenting the additional portion of patient data at the display device.

8. The method of claim 1, wherein at least part of the patient data is received from at least one of a client device associated with the patient and one or more sensors at the medical care site, wherein the client device comprises at least one of a smart phone and a smart wearable device, and wherein the one or more sensors comprise at least one of a wireless blood pressure sensor, a wireless heart rate sensor, a wireless body temperature sensor, a wireless pulse oximeter, a stethoscope, and an imaging sensor.

9. The method of claim 1, further comprising:
identifying an agenda for the patient consultation, the agenda being based on the patient data; and
presenting, at the display device and during the patient consultation, one or more agenda items from the agenda, the one or more agenda items being associated with a context of the patient consultation, the context comprising at least one of a consultation topic, a consultation activity, and a health status of the patient.

10. The method of claim 1, wherein the suggested consultation action is further based on the one or more agenda items, the method further comprising:
determining a completion of one or more activities associated with the one or more agenda items; and
presenting, at the display device, one or more different agenda items, the one or more different agenda items being based on at least one of the patient data, additional patient data collected during the patient consultation, and a result of the one or more activities associated with the one or more agenda items.

11. The method of claim 1, further comprising:
presenting, at the display device, a transcription of speech recognized during the patient consultation; and
presenting, at the display device, one or more workflow items determined for the patient consultation, the one or more workflow items being based on at least one of the patient data, additional patient data collected during the patient consultation, and the speech recognized during the patient consultation.

12. A system comprising:
memory; and
one or more processors coupled to the memory, the one or more processors being configured to:
present, at a display device at a medical care site, a suggested consultation action during a patient consultation, the suggested consultation action being based on patient data associated with a patient;
present, at the display device and during the patient consultation, a portion of patient data contextually relevant to the suggested consultation action, the patient consultation, and the patient; and
based on one or more measurements generated from the suggested consultation action, update the portion of patient data presented at the display device.

13. The system of claim 12, wherein the suggested consultation action comprises at least one of performing a medical test, performing a medical examination, and measuring a health metric via one or more medical devices.

14. The system of claim 13, wherein the medical test comprises at least one of a blood test, a scan, collecting and analyzing a specimen from the patient, a medical assessment, a genetic test, and a breathing test.

15. The system of claim 13, wherein the health metric comprises at least one of blood pressure, blood glucose levels, a pulse, a body temperature, and a body weight.

16. The system of claim 13, the one or more processors being configured to:
receive, from the one or more medical devices, at least one of a medical test result, a medical examination result and the health metric; and
present the portion of patient data in response to receiving the at least one of the medical test result, the medical examination result, and the health metric.

17. The system of claim 16, wherein the portion of patient data comprises additional patient data relevant to the suggested consultation action and the at least one of the medical test result, the medical examination result, and the health metric.

18. The system of claim 12, the one or more processors being configured to:
determine an additional portion of patient data, the additional portion of patient data being based on a current context of the patient consultation; and
present the additional portion of patient data at the display device.

19. The system of claim 12, wherein at least part of the patient data is received from at least one of a client device associated with the patient and one or more sensors at the medical care site, wherein the client device comprises at least one of a smart phone and a smart wearable device, and wherein the one or more sensors comprise at least one of a wireless blood pressure sensor, a wireless heart rate sensor, a wireless body temperature sensor, a wireless pulse oximeter, a stethoscope, and an imaging sensor.

20. The system of claim 12, the one or more processors being configured to:
identify an agenda for the patient consultation, the agenda being based on the patient data; and
present, at the display device and during the patient consultation, one or more agenda items from the agenda, the one or more agenda items being associated with a context of the patient consultation, the context comprising at least one of a consultation topic, a consultation activity, and a health status of the patient.
